(19) United States
(12) Patent Application Publication
(10) Pub. No.: US 2024/0228443 A1
(43) Pub. Date: Jul. 11, 2024

(54) NOVEL MANUFACTURING METHOD OF DAPRODUSTAT AND PRECURSORS THEREOF

(71) Applicant: GlaxoSmithKline Intellectual Property (No.2) Limited, Stevenage (GB)

(72) Inventors: Nicholas FLACO, Collegeville, PA (US); Qiang GUO, Collegeville, PA (US); John Jin LIM, Collegeville, PA (US); Kathryn Renee MEINTEL, King of Prussia, PA (US); Alexander Gordon O'BRIEN, Collegeville, PA (US)

(21) Appl. No.: 18/570,926

(22) PCT Filed: Jun. 18, 2021
(86) PCT No.: PCT/IB2021/055405

§ 371 Date: Dec. 15, 2023

(51) Int. Cl. C07D 239/62; A61K 31/515

(52) U.S. Cl. CPC C07D 239/62; A61K 31/515

(57) ABSTRACT

The present disclosure relates to a manufacturing process for daprodustat in which the level of an acyl impurity of Formula (II) is kept below 0.15% w/w in isolated daprodustat drug substance. Immediate release formulations of daprodustat containing a composition of daprodustat in which the level of the acyl impurity of Formula (II) is kept below 0.15% w/w relative to daprodustat drug substance are also disclosed. Medical uses of the immediate release formulation and dosage regimens are disclosed.

### FIELD OF THE INVENTION

The present disclosure relates to a manufacturing process for daprodustat in which the level of an acyl impurity of Formula (II) is kept below 0.15% w/w in isolated daprodustat drug substance. Immediate release formulations of daprodustat containing a composition of daprodustat in which the level of the acyl impurity of Formula (II) is kept below 0.15% w/w relative to daprodustat drug substance are also disclosed. Medical uses of the immediate release formulation and dosage regimens are disclosed.

### BACKGROUND TO THE INVENTION

Impurities can arise during the manufacturing process and/or storage of new drug substances. These include starting materials, intermediates, by-products, degradation products and reagents ligands and catalysts.

Given that impurities can produce pharmacological or toxic effects, each impurity identified by a manufacturer must be kept below a threshold level, termed its acceptance criteria. The acceptance criteria for a particular impurity reflects the effects associated with that impurity.

It is a regulatory requirement to produce a specification for a new drug substance. This must specify the acceptance criteria for identified impurities, the acceptance criteria for unspecified impurities and the acceptance criteria for total impurities.

Daprodustat is a prolyl hydroxylase inhibitor that is currently in development for the treatment of anemia due to chronic kidney disease. Daprodustat is the USAN, INN and JAN name for the compound N-((1,3-dicyclohexylhexahydro-2,4,6-trioxoyrimidin-5-yl)carbonyl)glycine. Small scale laboratory synthesis of daprodustat is disclosed in WO2007/150011.

### SUMMARY OF THE INVENTION

In a first aspect, the invention provides a process for preparing 1,3-dicyclohexylpyrimidine-2,4,6(1H,3H,5H)-trione comprising reacting 1,3-dicyclohexylurea with malonic acid, wherein the products of the reaction are 1,3-dicyclohexylpyrimidine-2,4,6(1H,3H,5H)-trione and a compound of formula (II)

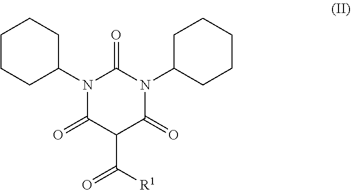

wherein R¹ is C₁₋₆alkyl, wherein the solvent system is selected to maintain the compound of formula (II) in solution at the temperature at which 1,3-dicyclohexylpyrimidine-2,4,6(1H,3H,5H)-trione is isolated, and wherein the reaction is terminated before the concentration of formula (II) exceeds its solubility in the solvent system employed.

In a second aspect, the invention provides a process for preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a step of preparing 1,3-dicyclohexylpyrimidine-2,4,6(1H,3H,5H)-trione as defined herein.

In a third aspect, the invention provides a composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof, wherein N-[(1,3-dicyclohexyl-6-hydroxy-2,4-dioxo-1,2,3,4-tetrahydro-5-pyrimidinyl)carbonyl]glycine or a pharmaceutically acceptable salt thereof is obtained according to a process as defined herein.

In a fourth aspect, the invention provides a process for preparing an immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a step of preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof as defined herein.

In a fifth aspect, the invention provides an immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof which composition comprises less than 0.15% w/w of a compound of formula (II) or a pharmaceutically acceptable salt thereof relative to daprodustat drug substance.

In a sixth aspect, the invention provides a compound of formula (II) or a pharmaceutically acceptable salt thereof:

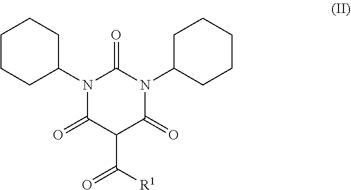

wherein R¹ is C₁₋₆alkyl.

In one particular embodiment, the invention provides a compound that is 5-acetyl-1,3-dicyclohexylpyrimidine-2,4,6(1H,3H,5H)-trione.

In further aspects, the invention provides medical uses of the immediate release formulation defined herein.

### DESCRIPTION OF DRAWINGS/FIGURES

FIG. 1 shows the effect of imidazole (0.5% w/w) on progress of the Stage 1 reaction. FIG. 1A shows the rate of intermediate consumption. FIG. 1B shows the rate of product (1,3-dicyclohexylpyrimidine-2,4,6(1H,3H,5H)-trione) formation. FIG. 1C shows the rate of formation of 1,3-dicyclohexyl-2H-pyrano[2,3-d]pyrimidine-2,4,5,7(1H,3H,6H)-tetraone and FIG. 1D shows the rate of formation of 5-acetyl-1,3-dicyclohexylpyrimidine-2,4,6(1H,3H,5H)-trione. Solid lines show the reaction rate in the presence of the imidazole additive. Dashed lines show the reaction rate without additive.

FIG. 2 shows the effect of effect of N,N-dimethylaminopyridine (0.9% w/w; equivalent to 1.65 mol % relative to N,N-dicyclohexylurea) on progress of the Stage 1 reaction. FIG. 2A shows the rate of intermediate consumption. FIG. 2B shows the rate of product (1,3-dicyclohexylpyrimidine-2,4,6(1H,3H,5H)-trione) formation. FIG. 2C shows the rate of formation of 1,3-dicyclohexyl-2H-pyrano[2,3-d]pyrimidine-2,4,5,7(1H,3H,6H)-tetraone and FIG. 2D shows the rate of formation of 5-acetyl-1,3-dicyclohexylpyrimidine-2,4,6(1H,3H,5H)-trione. Solid lines show the reaction rate in the presence of the imidazole additive. Dashed lines show the reaction rate without additive.

FIG. 3 shows the effect of varying amounts (in % w/w) of imidazole in N,N-dicyclohexylurea on progress of the Stage 1 reaction. FIG. 3A shows the rate of intermediate consumption. FIG. 3B shows the rate of product (1,3-dicyclohexylpyrimidine-2,4,6(1H,3H,5H)-trione) formation. FIG. 3C shows the rate of formation of 1,3-dicyclohexyl-2H-pyrano[2,3-d]pyrimidine-2,4,5,7(1H,3H,6H)-tetraone and FIG. 3D shows the rate of formation of 5-acetyl-1,3-dicyclohexylpyrimidine-2,4,6(1H,3H,5H)-trione. Solid lines show the reaction rate in the presence of the imidazole additive. Dashed lines show the reaction rate without additive.

FIG. 4 is a Half-Normal Plot summarizing the model for the rate of formation of 5-acetyl-1,3-dicyclohexylpyrimidine-2,4,6(1H,3H,5H)-trione. CD show the combined effects of imidazole and quantity of acetic acid and CE shows the combined effect of imidazole and reaction temperature.

FIGS. 5A and B are interaction plots. FIG. 5A shows the effect of the amount (in % w/w) of imidazole in N,N-dicyclohexylurea upon the initial rate of formation of 5-acetyl-1,3-dicyclohexylpyrimidine-2,4,6(1H,3H,5H)-trione of in the presence of 2 volumes acetic acid (squares) and 4 volumes of acetic acid (triangles). FIG. 5B shows the effect of the amount (in % w/w) of imidazole in N,N-dicyclohexylurea upon the initial rate of formation of 5-acetyl-1,3-dicyclohexylpyrimidine-2,4,6(1H,3H,5H)-trione at reaction temperatures of 50° C. (squares) and 60° C. (triangles).

### DETAILED DESCRIPTION OF THE INVENTION

### Manufacture

Daprodustat is the USAN, INN and JAN name for the compound N-((1,3-dicyclohexylhexahydro-2,4,6-trioxopyrimidin-5-yl)carbonyl)glycine (the IUPAC name for this compound is N-[(1,3-Dicyclohexylhexahydro-2,4,6-trioxopyrimidin-5-yl)carbonyl]glycine). Daprodustat exhibits keto/enol tautomerism and can also be named N-[(1,3-dicyclohexyl-6-hydroxy-2,4-dioxo-1,2,3,4-tetrahydro-5-pyrimidinyl)carbonyl]glycine. Where claims refer to N-[(1,3-dicyclohexyl-6-hydroxy-2,4-dioxo-1,2,3,4-tetrahydro-5-pyrimidinyl)carbonyl]glycine, all tautomers of N-[(1,3-dicyclohexyl-6-hydroxy-2,4-dioxo-1,2,3,4-tetrahydro-5-pyrimidinyl)carbonyl]glycine, including mixtures thereof, are intended to be encompassed within the scope of the invention.

The free acid and metal salts of daprodustat may be manufactured according to the general synthetic scheme shown below:

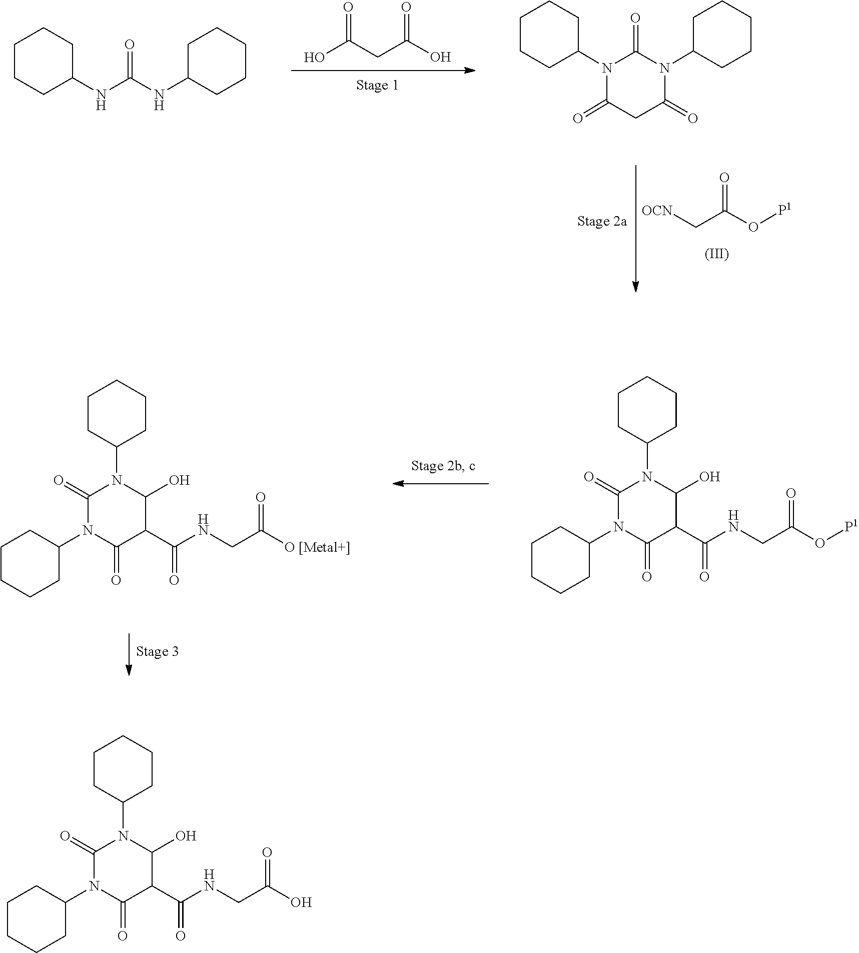

wherein P¹ is a suitable protecting group, such as C₁₋₆alkyl, benzyl or vinyl.

The reaction of Stage 1 requires the use of a dehydrating agent, for example a compound of formula (I):

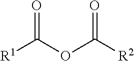

Wherein R¹ is C₁₋₆alkyl and R² is C₁₋₆alkyl.

In addition to the desired reaction in Stage 1, a side reaction occurs leading to the production of an acyl impurity of formula (II):

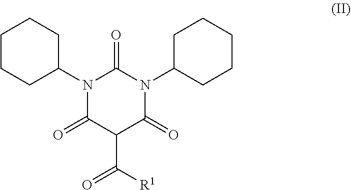

Additionally, the impurity of formula (II) may be generated in Stage 1 and Stage 2 by reaction with a carboxylic acid of formula R¹CO₂H, where this carboxylic acid is present (e.g. as a solvent).

In one embodiment, R¹ is methyl, such that the compound of formula (II) is 5-acetyl-1,3-dicyclohexylpyrimidine-2,4,6(1H,3H,5H)-trione.

As demonstrated in Example 1, the impurity of Formula (II) is not readily purged in Stages 2 and 3 of the process. Example 4 teaches that, where the concentration of the impurity of Formula (II) is below its solubility in the solvent system employed in Stage 1, the impurity can be purged effectively. Example 4, specifically demonstrates purging of a compound of formula (II) that is 5-acetyl-1,3-dicyclohexylpyrimidine-2,4,6(1H,3H,5H)-trione. 5-Acetyl-1,3-dicyclohexylpyrimidine-2,4,6(1H,3H,5H)-trione has a solubility of 43.7 mg/ml in 29.5% v/v acetic anhydride-acetic acid (the solvent system employed for Stage 1 in example 4) at 15° C. (the isolation temperature used in Example 4). The experiment in Example 4 demonstrated that 45.2 mg/ml 5-acetyl-1,3-dicyclohexylpyrimidine-2,4,6(1H,3H,5H)-trione could be completely purged during isolation of the product of Stage 1. In other words, complete purging was observed where the concentration of 5-acetyl-1,3-dicyclohexylpyrimidine-2,4,6(1H,3H,5H)-trione approximates the solubility of this compound in the solvent system at the isolation temperature.

The skilled person would expect other compounds of formula (II) to be purged during isolation of the product of Stage 1 in the situation where (as in Example 4) they are substantially dissolved in the solvent system. In other words, the skilled person would realise that the concentration of the compound of Formula (II) at the time the reaction is terminated must be no greater than the solubility limit for the solvent system at the isolation temperature. In view of this, the skilled reader would appreciate the necessity of terminating the reaction whilst the concentration of the compound of formula (II) was below the solubility limit for the solvent system at the isolation temperature.

Accordingly, in a first aspect, the invention provides a process for preparing 1,3-dicyclohexylpyrimidine-2,4,6(1H,3H,5H)-trione comprising reacting 1,3-dicyclohexylurea with malonic acid, wherein the products of the reaction are 1,3-dicyclohexylpyrimidine-2,4,6(1H,3H,5H)-trione and a compound of formula (II)

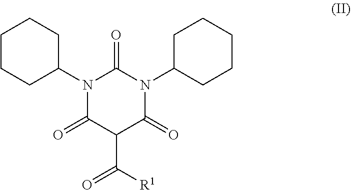

wherein R¹ is C₁₋₆alkyl, wherein the solvent system is selected to maintain the compound of formula (II) in solution at the temperature at which 1,3-dicyclohexylpyrimidine-2,4,6(1H,3H,5H)-trione is isolated, and wherein the reaction is terminated before the concentration of formula (II) exceeds its solubility in the solvent system employed.

It will be apparent that the point at which the reaction is terminated will depend upon both the compound of formula (II), the solvent system employed and the isolation temperature selected.

Where the compound of formula (II) is 5-acetyl-1,3-dicyclohexylpyrimidine-2,4,6(1H,3H,5H)-trione, the solvent system is 29.5% v/v acetic anhydride-acetic acid, and the isolation temperature is 15° C., the reaction should be terminated before the concentration of 5-acetyl-1,3-dicyclohexylpyrimidine-2,4,6(1H,3H,5H)-trione is 45.2 mg/ml.

Solubility studies such as those described in Example 4 can be used to determine the point at which the reaction is terminated for any particular compound of formula (II), solvent system and isolation temperature.

HPLC can be used to monitor reaction progress. Detection at 210 nm can be used to detect both the product of Stage 1 and the compound of formula (II). As shown in Example 4, 1,3-dicyclohexylpyrimidine-2,4,6(1H,3H,5H)-trione was completely purged when the % area by HPLC was 45.6. Accordingly, HPLC monitoring can be used as an alternative to monitoring concentration of 5-acetyl-1,3-dicyclohexylpyrimidine-2,4,6(1H,3H,5H)-trione to identify the point at which the reaction of Stage 1 should be terminated.

In one embodiment where the compound of formula (II) is 5-acetyl-1,3-dicyclohexylpyrimidine-2,4,6(1H,3H,5H)-trione and the reaction conditions used to prepare 1,3-dicyclohexylpyrimidine-2,4,6(1H,3H,5H)-trione and 5-acetyl-1,3-dicyclohexylpyrimidine-2,4,6(1H,3H,5H)-trione used 1,3-dicyclohexylurea (1.0 wt), malonic acid (1.3 wt), acetic acid (2.7 wt) and acetic anhydride (6.6 eq) and the isolation temperature is 15° C., the reaction should be terminated before the percentage of 5-acetyl-1,3-dicyclohexylpyrimidine-2,4,6(1H,3H,5H)-trione exceeds 45% by area of the products of the reaction. HPLC monitoring can be used to identify the point at which the reaction of Stage 1 should be terminated for other compounds of formula (II). The percentage by area of the compound of formula (II) when this is sufficiently dissolved in the appropriate solvent system can readily be determined by HPLC. Where the concentration of the compound of formula (II) is at its solubility limit for the solvent system and isolation temperature, this percentage by area approximates the point at which the Stage 1 reaction should be terminated.

The Stage 1 reaction takes place in the presence of a suitable solvent system. The solvent system is selected to maintain the compound of formula (II) in solution and the product of the Stage 1 reaction (1,3-dicyclohexylpyrimidine-2,4,6(1H,3H,5H)-trione) substantially not in solution at the temperature at which 1,3-dicyclohexylpyrimidine-2,4,6(1H,3H,5H)-trione is isolated, such that the compound of formula (II) can be removed by filtration (in the filtrate). In one embodiment, the solvent is a mixture of a carboxylic acid of formula R¹CO₂H. The dehydrating agent of formula (I) may also form part of the solvent system. In one embodiment, the solvent system is a mixture of acetic acid and the dehydrating agent, acetic anhydride.

The reaction may be terminated by any convenient method. In one embodiment, the reaction is mixture is cooled, for example to 15-25° C. In another embodiment, the reaction is terminated by filtration. Filtration terminates the reaction by removal of starting materials and impurities (including the compound of formula II), and results in a filter cake. The filter cake may be washed to purify the product of the reaction and to remove traces of solvents that are incompatible with subsequent steps or which lead to undesirable side reactions, for example side reactions leading to the compound of formula (II).

In a particular embodiment, where a carboxylic acid of formula R¹CO₂H is used in Stage 1, the filter cake is washed to reduce the levels of this carboxylic acid to not greater than 0.1% (w/w). Levels of the carboxylic acid of formula R¹CO₂H may be measured by gas chromatography. Calibration may be used to covert a gas chromatography peak for the carboxylic acid of formula R¹CO₂H to its weight. In a particular embodiment, the filter cake is washed with a solvent in which the compound of formula (II) significantly more soluble than 1,3-dicyclohexylpyrimidine-2,4,6(1H,3H,5H)-trione at the temperature employed. Washes may also be employed simply to displace other solvents.

In one embodiment, the filter cake is washed with not less than 2 volumes of a solvent other than a carboxylic acid of formula R¹CO₂H.

In a particular embodiment in which the compound of formula (II) is 5-acetyl-1,3-dicyclohexylpyrimidine-2,4,6(1H,3H,5H)-trione, the solvent system is 29.5% v/v acetic anhydride-acetic acid, and the isolation temperature is 15° C., the filter cake is washed with 2 volumes acetic acid and 2 volumes water. The first wash is used to displace the reaction liquors in the filter equipment, and the second wash is used to displace the acetic acid.

This invention teaches how to control the compound of formula (II) in the final product, daprodustat. As described above, the key control is to terminate the Stage 1 reaction at a point at which the compound of formula (II) can be effectively purged. By meeting this, the compound of formula (II) will not appear in daprodustat drug substance above the 0.15% threshold set by regulatory authorities.

In addition to this, the inventors have identified how to limit the production of the compound of formula (II). It will be appreciated that, by reducing the production of the compound in formula (II) in Stage 1, a greater yield of the product can be achieved before the reaction must be terminated. Specifically, the inventors have identified that the product specification of the starting material 1,3-dicyclohexylurea and in particular the content of imidazole in the starting material has major impact on the production of the compound of formula (II). Imidazole is a by-product of the manufacturing process for 1,3-dicyclohexylurea starting from N,N′-carbonyldiimidazole. It is purged during Stage 1, but also catalyses the formation of the compound of formula (II). Examples 2 and 3 demonstrate that the imidazole content of the starting material 1,3-dicyclohexylurea has a significant impact upon the level of the compound of formula (II) after Stage 1. Accordingly, in one embodiment the 1,3-dicyclohexylurea starting material contains not greater than 0.05% (w/w) imidazole. Whilst a small effect on formation of the compound of formula (II) is observed with this level of imidazole, reduction of the imidazole content below this level is practically difficult to achieve for suppliers.

Additionally, the inventors identified an impact of the Stage 1 reaction temperature upon the rate of formulation of the compound of formula (II). In one embodiment, the Stage 1 reaction is conducted at a temperature between 50-60° C. In a more particular embodiment, the Stage 1 reaction is conducted at 50° C.

In one aspect, the invention provides a process for preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof comprising a step of preparing 1,3-dicyclohexylpyrimidine-2,4,6(1H,3H,5H)-trione as defined herein.

Subsequent steps of the process may be conducted as shown in general synthetic scheme shown above.

Stage 2a comprises reaction with a compound of formula (III) such as ethyl 2-isocyanatoacetate in the presence of a suitable base such as triethylamine in a suitable solvent such as tetrahydrofuran.

Stage 2b comprises reaction with a metal hydroxide to form the appropriate salt. For example, treatment with aqueous potassium hydroxide results in the formation of the potassium salt. Treatment with aqueous sodium hydroxide results in formation of the sodium salt.

Stage 2c is an evaporation step aimed at reducing levels of solvents used in earlier stages, e.g. tetrahydrofuran.

Stage 3 comprises treatment with an acid, such as aqueous hydrochloric acid in a suitable solvent such as acetone.

Example 1 describes the impact of acetic acid in Stage 2 upon the levels of 5-acetyl-1,3-dicyclohexylpyrimidine-2,4,6(1H,3H,5H)-trione in daprodustat. Table 4 showed that levels of 0.1% acetic acid can be tolerated in Stage 2. Accordingly, in one embodiment, the levels of a carboxylic acid of formula R¹CO₂H in 1,3-dicyclohexylpyrimidine-2,4,6(1H,3H,5H)-trione are less than or equal to 0.1% (w/w) immediately prior to reaction with a compound of formula (III).

In one embodiment, the product of the process is a pharmaceutically acceptable salt of N-[(1,3-dicyclohexyl-6-hydroxy-2,4-dioxo-1,2,3,4-tetrahydro-5-pyrimidinyl)carbonyl]glycine.

In one embodiment, the product of the process is N-[(1,3-dicyclohexyl-6-hydroxy-2,4-dioxo-1,2,3,4-tetrahydro-5-pyrimidinyl)carbonyl]glycine free acid.

In one embodiment, the product of the process (N-[(1,3-dicyclohexyl-6-hydroxy-2,4-dioxo-1,2,3,4-tetrahydro-5-pyrimidinyl)carbonyl]glycine or a pharmaceutically acceptable salt thereof) is in crystalline form.

In one embodiment, the product of the process (N-[(1,3-dicyclohexyl-6-hydroxy-2,4-dioxo-1,2,3,4-tetrahydro-5-pyrimidinyl)carbonyl]glycine free acid is in crystalline form.

In a particular embodiment, the product of the process (N-[(1,3-dicyclohexyl-6-hydroxy-2,4-dioxo-1,2,3,4-tetrahydro-5-pyrimidinyl)carbonyl]glycine free acid is a non-solvated crystalline form referred to as CS1. Form CS1 has an X-ray powder diffraction pattern that has characteristic peaks at 2theta values of 6.4°±0.2°, 7.5°±0.2°, and 7.9°≅0.2° using CuKα radiation. In a more particular embodiment, the X-ray powder diffraction pattern of form CS1 has one or more additional characteristic peaks at 2theta values of 17.2°±0.2°, 21.0°±0.2°, 24.0°±0.2°, and 19.3°±0.2° using CuKα radiation. Form CS1 has an endothermic peak at around 242° C. as measured by differential scanning calorimetry using a heating rate of 10° C. min and a purge gas of nitrogen.

In another embodiment, the product of the process (N-[(1,3-dicyclohexyl-6-hydroxy-2,4-dioxo-1,2,3,4-tetrahydro-5-pyrimidinyl)carbonyl]glycine free acid is a non-solvated crystalline form referred to as CS9. Form CS9 has an X-ray powder diffraction pattern that has characteristic peaks at 2theta values of 4.6°±0.2°, 6.6°±0.2°, and 21.1°±0.2° using CuKα radiation. In a more particular embodiment, the X-ray powder diffraction pattern for form CS9 has one or more additional characteristic peaks at 2theta values of 9.4°±0.2°, 20.2°±0.2°, and 24.2°±0.2° using CuKα radiation.

Forms CS1 and CS9 may be crystallised from the free acid according to processes described in WO2019052133.

In another embodiment, the product of the process (N-[(1,3-dicyclohexyl-6-hydroxy-2,4-dioxo-1,2,3,4-tetrahydro-5-pyrimidinyl)carbonyl]glycine free acid is a crystalline form referred to as Form 3. Form 3 has an X-ray powder diffraction pattern having peaks at 2-theta values of 4.5°±0.2°, 5.6°±0.2°, 9.0°±0.2° and 16.8°±0.2° using CuKα radiation. In a more particular embodiment, the X-ray powder diffraction pattern of Form 3 has one or more additional characteristic peaks at 2-theta values selected from 8.5°±0.2°, 11.2°±0.2°, 20.6°±0.2° and 24.7°±0.2° using CuKα radiation and/or a DSC endothermic peak with T onset at about 245.3° C.

In another embodiment, the product of the process (N-[(1,3-dicyclohexyl-6-hydroxy-2,4-dioxo-1,2,3,4-tetrahydro-5-pyrimidinyl)carbonyl]glycine free acid is a crystalline form referred to as Form 4. Form 4 has an X-ray powder diffraction pattern having peaks at 2-theta values of 7.2°±0.2°, 11.5°±0.2°, 21.7°±0.2°, 22.9°±0.2°, 23.3°±0.2° and 25.8°±0.2° using CuKα radiation. In a more particular embodiment, the X-ray powder diffraction pattern of Form 4 has one or more additional characteristic peaks at 2-theta values selected from 6.3°±0.2°, 12.9°±0.2°, 16.5°±0.2°, 18.1°±0.2° and 19.7°±0.2° using CuKα radiation, and/or a DSC endothermic peak with T onset at about 243.9° C.

Forms 3 and 4 may be crystallised as described in WO2020102302.

In another embodiment, the product of the process (N-[(1,3-dicyclohexyl-6-hydroxy-2,4-dioxo-1,2,3,4-tetrahydro-5-pyrimidinyl)carbonyl]glycine free acid is a crystalline form referred to as form M. Form M has an X-ray powder diffraction pattern that has characteristic peaks at 2theta values of 4.7°±0.2°, 6.5°±0.2°, and 6.8°±0.2° using CuKα radiation. Form M may be crystallised as described in WO2021031102.

In one aspect, the invention provides a composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof, wherein N-[(1,3-dicyclohexyl-6-hydroxy-2,4-dioxo-1,2,3,4-tetrahydro-5-pyrimidinyl)carbonyl]glycine or a pharmaceutically acceptable salt thereof is obtained according to a process described herein. In a related aspect, the invention provides a composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof, which composition comprises less than 0.15% w/w of the compound of formula (II) or a pharmaceutically acceptable salt thereof. In one embodiment, the composition comprises between 0.01-0.15% w/w of the compound of formula (II) or a pharmaceutically acceptable salt thereof.

In another aspect, the invention provides a compound of formula (II) or a pharmaceutically acceptable salt thereof:

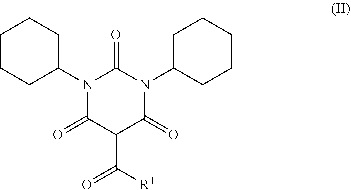

wherein R¹ is C₁₋₆alkyl.

In one embodiment, R¹ is methyl in the compound of formula (II) or a pharmaceutically acceptable salt thereof.

In one embodiment, the invention provides a compound that is: 5-acetyl-1,3-dicyclohexylpyrimidine-2,4,6(1H,3H,5H)-trione.

The compound of formula (II) exhibits keto/enol tautomerism and the specific compound of formula (II) that is 5-acetyl-1,3-dicyclohexylpyrimidine-2,4,6(1H,3H,5H)-trione can also be named 5-acetyl-1,3-dicyclohexyl-6-hydroxypyrimidine-2,4(1H,3H)-dione. Where claims refer to a compound of formula (II) or 5-acetyl-1,3-dicyclohexylpyrimidine-2,4,6(1H,3H,5H)-trione, all tautomers, including mixtures thereof, are intended to be encompassed within the scope of the invention.

### Pharmaceutical Compositions

In another aspect, the invention provides a process for preparing an immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a step of preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof as defined herein. In one embodiment, said N-[(1,3-dicyclohexyl-6-hydroxy-2,4-dioxo-1,2,3,4-tetrahydro-5-pyrimidinyl)carbonyl]glycine or a pharmaceutically acceptable salt thereof is N-[(1,3-dicyclohexyl-6-hydroxy-2,4-dioxo-1,2,3,4-tetrahydro-5-pyrimidinyl)carbonyl]glycine free acid. In a more particular embodiment, N-[(1,3-dicyclohexyl-6-hydroxy-2,4-dioxo-1,2,3,4-tetrahydro-5-pyrimidinyl)carbonyl]glycine free acid is in crystalline form. In an alternative embodiment, said N-[(1,3-dicyclohexyl-6-hydroxy-2,4-dioxo-1,2,3,4-tetrahydro-5-pyrimidinyl)carbonyl]glycine or a pharmaceutically acceptable salt thereof is a pharmaceutically acceptable salt of N-[(1,3-dicyclohexyl-6-hydroxy-2,4-dioxo-1,2,3,4-tetrahydro-5-pyrimidinyl)carbonyl]glycine, for example the sodium or potassium salt.

In another aspect, the invention provides an immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof obtained by a process described herein.

In one aspect, the invention provides an immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof which composition comprises less than 0.15% w/w of a compound of formula (II) or a pharmaceutically acceptable salt thereof, relative to levels of N-[(1,3-dicyclohexyl-6-hydroxy-2,4-dioxo-1,2,3,4-tetrahydro-5-pyrimidinyl)carbonyl]glycine or a pharmaceutically acceptable salt thereof.

In one aspect, the immediate release formulation of the invention comprises a composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof which composition comprises less than 0.15% w/w of a compound of formula (II) or a pharmaceutically acceptable salt thereof.

In one embodiment, the immediate release formulation of the invention comprises between 0.01-0.15%/w of a compound of formula (II) or a pharmaceutically acceptable salt thereof.

In one embodiment, the compound of formula (II) or a pharmaceutically acceptable salt thereof is 5-acetyl-1,3-dicyclohexylpyrimidine-2,4,6(1H,3H,5H)-trione or a pharmaceutically acceptable salt thereof. In one embodiment, the compound of formula (II) or a pharmaceutically acceptable salt thereof is 5-acetyl-1,3-dicyclohexylpyrimidine-2,4,6(1H,3H,5H)-trione.

In one embodiment, the immediate release formulation of the invention is a tablet.

In one embodiment, an immediate release tablet of N-[(1,3-dicyclohexyl-6-hydroxy-2,4-dioxo-1,2,3,4-tetrahydro-5-pyrimidinyl)carbonyl]glycine or a pharmaceutically acceptable salt thereof is a tablet comprising from 1 to 8 mg (measured as the free acid) of N-[(1,3-dicyclohexyl-6-hydroxy-2,4-dioxo-1,2,3,4-tetrahydro-5-pyrimidinyl)carbonyl]glycine or a pharmaceutically acceptable salt thereof that meets the following dissolution criteria:
1. A mean (based on at least 12 tablets) of 85% or more of the of N-[(1,3-dicyclohexyl-6-hydroxy-2,4-dioxo-1,2,3,4-tetrahydro-5-pyrimidinyl)carbonyl]glycine free acid contained in the tablet dissolves within 45 minutes or less using United States Pharmacopeia (USP) Apparatus 2 with a rotational speed of 50±2 rpm and a dissolution volume of 500±5 mL for tablets containing <2 mg of N-[(1,3-dicyclohexyl-6-hydroxy-2,4-dioxo-1,2,3,4-tetrahydro-5-pyrimidinyl)carbonyl]glycine or a pharmaceutically acceptable salt thereof (measured as the free acid) and 900±9 mL for tablets containing ≥2 mg of N-[(1,3-dicyclohexyl-6-hydroxy-2,4-dioxo-1,2,3,4-tetrahydro-5-pyrimidinyl)carbonyl]glycine or a pharmaceutically acceptable salt thereof (measured as the free acid) in a pH 6.8 buffer or Simulated Intestinal Fluid USP without enzymes.

In one embodiment, the dissolution profile of an immediate release tablet comprising from 1 to 8 mg (measured as the free acid) of N-[(1,3-dicyclohexyl-6-hydroxy-2,4-dioxo-1,2,3,4-tetrahydro-5-pyrimidinyl)carbonyl]glycine or a pharmaceutically acceptable salt thereof or a pharmaceutically acceptable salt thereof using United States Pharmacopeia (USP) Apparatus 2 under the conditions specified above must additionally exhibit an f2 value ≥50 compared to a tablet as described in Example 5 containing the same dose of active pharmaceutical ingredient. In one embodiment, the tablet of Example 5 was compacted using a main compaction pressure of 200-290 MPa, more particularly 240-260 MPa and even more particularly, about 250 MPa.

The immediate release tablet of the invention may comprise from 1 to 8 mg (measured as the free acid) daprodustat or a pharmaceutically acceptable salt thereof which has a tablet tensile strength of greater or equal to 1.7 MPa following compaction of the tablet core at a pressure in the range of 200 to 290 MPa. In more particular embodiments, the tablet tensile strength is greater than or equal to 1.75, 1.8, 1.9 or 2.0 MPa following compaction of the tablet core at a pressure in the range of 200 to 290 MPa.

In one embodiment, the immediate release tablet of the invention comprises a compartment containing daprodustat or a pharmaceutically acceptable salt thereof in an amount up to 5% based on the weight of the free acid, where the compartment does not contain a glidant. In one embodiment, the compartment contains the non solvated crystalline form of daprodustat free acid. In a particular embodiment, the non-solvated crystalline form of daprodustat free acid is form CS1.

In one embodiment, the tablet is a monolithic tablet consisting of a single compartment of uniform composition that is optionally film coated.

In an alternative embodiment, the tablet contains granules dispersed in an extragranular space and is optionally film coated. The granular and extragranular compositions may be different and form separate compartments. In one embodiment, the granular compartment is the compartment containing daprodustat or a pharmaceutically acceptable salt thereof (for example the non-solvated crystalline form of daprodustat free acid) and no glidant.

In one embodiment, the intragranular compartment comprises the crystalline form of non-solvated daprodustat free acid, a diluent, a binder and a disintegrant and no glidant. For the avoidance of doubt, more than one diluent, binder of disintegrant may be included. In one embodiment, the intragranular compartment consists of the crystalline form of non-solvated daprodustat free acid, one or more diluents, a binder and a disintegrant and no glidant.

In one embodiment, the extragranular compartment comprises a diluent, a disintegrant, a lubricant, and optionally a glidant. For the avoidance of doubt, more than one diluent, disintegrant, lubricant or glidant may be included. In one embodiment, the extragranular compartment consists of one or more diluents, a disintegrant, a lubricant, and optionally a glidant.

Suitable diluents include lactose, sucrose, dextrose, mannitol, sorbitol, starch (e.g. corn starch, potato starch, and pre-gelatinized starch), cellulose and its derivatives (e.g., microcrystalline cellulose), calcium sulfate, and dibasic calcium phosphate. In one embodiment, the diluent is not lactose.

Suitable binders include starch (e.g., corn starch, potato starch, and pre-gelatinized starch), hypromellose, gelatin, acacia, sodium alginate, alginic acid, tragacanth, guar gum, povidone, and cellulose and its derivatives (e.g. microcrystalline cellulose).

Suitable disintegrants include crospovidone, sodium starch glycolate, croscarmellose sodium, alginic acid, and sodium carboxymethyl cellulose.

Suitable lubricants include stearic acid, magnesium stearate, calcium stearate, and talc.

Glidants include colloidal silicon dioxide, talc, starch and magnesium stearate. In one embodiment, the glidant is colloidal silicon dioxide or magnesium stearate. In one embodiment, the glidant is silica. In one embodiment, the glidant is colloidal silicon dioxide.

In one embodiment, the immediate release tablet consists of:
a) intragranular components comprising the crystalline form of non-solvated daprodustat free acid, a diluent, a binder and a disintegrant; and
b) extragranular components comprising a diluent, a disintegrant, a lubricant, and optionally a glidant;
wherein the tablet is optionally coated.

In a more particular embodiment, the immediate release tablet consists of:
a) intragranular components consisting of the crystalline form of non-solvated daprodustat free acid and one or more diluents, one or more binders and one or more disintegrants; and
b) extragranular components comprising a diluent, a disintegrant, a lubricant, and optionally a glidant;
wherein the tablet is optionally coated.

A coating may be applied to the tablet core. An example of a commercially available coating is “OPADRY OY-S-28876 WHITE”. Coloured coatings are also commercially available.

In one embodiment, the immediate release tablet contains up to 76% by weight of intragranular components based on the weight of an uncoated tablet.

In one embodiment, the immediate release tablet comprises an intragranular compartment and an extragranular compartment wherein:
a. the intragranular components comprise:
   i. 1 to 10 mg of the crystalline form of non-solvated daprodustat free acid;
   ii. about 5 wt % hypromellose;
   iii. about 1.5 wt % croscarmellose sodium; and
   iv. mannitol and microcryrstalline cellulose in a weight ratio from about 2.2 to about 3.6 (e.g., from about 2.3 to about 3.5, or about 2.25);
b. the extragranular components comprise, based on the total weight of the extragranular components:
   i. about 12 wt % croscarmellose sodium;
   ii. about 4 wt % magnesium stearate;
   iii. about 1.5% colloidal silica; and
   iv. mannitol and microcryrstalline cellulose in a weight ratio from about 0.3 to about 3 (e.g. about 2).
In a particular embodiment, the tablet comprises about 1, 2 or 4 mg daprodustat and has a core tablet weight of about 150 mg. In another embodiment, the tablet comprises about 6 or 8 mg daprodustat and has a core tablet weight of about 300 mg. The tablets described herein may be optionally film coated.

In one embodiment, the immediate release tablet does not comprise lactose.

### Medical Use

The immediate release formulation of the invention may be used in therapy, more particularly in the treatment of anemia. In a particular embodiment, the immediate release formulation of the invention may be used in the treatment of anemia due to chronic kidney disease (also known as renal anemia), anemia in patients with cancer receiving chemotherapy (including myelosuppressive or platinum containing chemotherapy), anemia in zidovudine-treated HIV-infected patients and anemia due to rheumatoid arthritis. In one embodiment, the immediate release formulation of the invention may be administered to patients receiving elective orthopaedic surgery.

Accordingly, in one embodiment, the invention provides the immediate release formulation of the invention for use in therapy.

In another embodiment, the invention provides the immediate release formulation of the invention for use in a method of treating anemia due to chronic kidney disease.

In yet another embodiment, the invention provides use of a composition of daprodustat or a pharmaceutically acceptable salt thereof which composition comprises less than 0.15% w/w of a compound of formula (II) or a pharmaceutically acceptable salt thereofin the manufacture of the immediate release formulation of the invention for use in the treatment of anemia due to chronic kidney disease.

In another embodiment, the invention provides a method for the treatment of anemia due to chronic kidney disease in a subject in need thereof, comprising administering to said subject the immediate release formulation of the invention.

Suitably, the subject is a mammal. In a particular embodiment, the subject is human.

In more particular embodiments, the subject having anemia due to chronic kidney disease may be receiving dialysis, for example haemodialysis or peritoneal dialysis. In another embodiment, the subject may be iron deficient (TSAT≤20% and/or serum ferritin≤100 ng/ml) and additionally receiving supplemental iron therapy.

In a further embodiment, the invention provides a dosage regimen for the treatment of anemia due to chronic kidney disease which aims to maintain haemoglobin in the range 10 to 12 g/dL and provide a safe increase in haemoglobin levels where haemoglobin levels are below this. The dose is modified based on the concentration of haemoglobin determined at clinical visits. Haemoglobin concentration may be measured by known methods for example HemoCue.

In one aspect, the invention provides a dosage regimen for the treatment of anemia due to chronic kidney disease for patients wherein the immediate release formulation of the invention is administered once daily at a dose of either 1 mg, 2 mg, 4 mg, 6 mg, 8 mg, 12 mg, 16 mg or 24 mg and wherein the dose is increased or decreased by one dose step based on the haemoglobin concentration of the patient to maintain the haemoglobin concentration of the patient within the range 10-12 g/dL. In one embodiment, the dose is increased or decreased by one dose step based on the haemoglobin concentration of the patient to maintain the haemoglobin concentration of the patient within the range 10-11 g/dL. In one embodiment, the dose is increased or decreased by one dose step based on the haemoglobin concentration of the patient to maintain the haemoglobin concentration of the patient at a target of 10 g/dL.

In particular embodiments, the haemoglobin concentration of the patient is monitored at least once every three months. In more particular embodiments, the haemoglobin concentration of the patient is monitored monthly or every four weeks. The skilled person will appreciate that monitoring may be more frequent when treatment is initiated, with the frequency of monitoring decreasing once the haemoglobin concentration of the patient has stabilised within the target range/at the target (10 to 12 g/dL or 10 to 11 g/dL or 10 g/dL).

In embodiments when there is a rapid increase in the haemoglobin concentration of the patient (e.g., exceeding 2.0 g/dL within 4 weeks), the dose is reduced by one dose step or interrupted.

In embodiments where the haemoglobin concentration of the patient exceeds the top end of the target range, the dose is interrupted until the haemoglobin concentration is in target range, and treatment is re-started at one dose level lower.

Clinical judgement is also important in dose increases and reductions. In embodiments where the patient is above the target range and at risk of thromboembolism (e.g. where a patient has had a stroke), the dose is reduced by one dose step or interrupted. In embodiments where the patient is exhibiting symptoms of anemia, the dose is increased by one dose step.

In one embodiment, the patient is not on dialysis. In another embodiment, the patient is on dialysis (e.g., haemodialysis or peritoneal dialysis).

In embodiments where the patient is not on dialysis and the patient has previously been treated with an erythropoiesis stimulating agent (ESA), starting doses are based on prior ESA dosage. In embodiments where the patient is not on dialysis and the patient has previously been treated with an erythropoiesis stimulating agent (ESA), starting doses are based on the patient's haemoglobin concentration. Table 1 sets out suitable starting doses.

TABLE 1

| Patients Switching from ESA Therapy | | | |
|---|---|---|---|
| Current ESA Dosage | | | |
| | | methoxy | |
| Epoetin alfa | Darbepoetin alfa | PEG-epoetin | |
| IV | SC/IV | beta SC/IV | Daprodustat |
| (units/week) | (mcg/4 week) | (mcg /month) | once daily |
| 1500 to 2000 | 20 to 30 | 30 to 40 | 1 mg |
| >2000 to <20,000 | >30 to 300 | >40 to 360 | 2 mg |
| ≥20,000 | >300 | >360 | 4 mg |

| | |
|---|---|
| Patients Not Receiving ESA Therapy | |
| Haemoglobin level (g/dL) | |
| <9 | 4 mg |
| ≥9 | 2 mg |

¹ESA: Erythropoiesis-stimulating agent

A dosage regimen for treatment of anemia due to chronic kidney disease to maintain haemoglobin concentration in the range 10-11 g/dL is provided, wherein the immediate release tablet of the invention is administered once daily at one of the following doses: 1, 2, 4, 6, 8, 12, 16 and 24 mg (dose of free acid), and wherein:
a) where the haemoglobin concentration ≥12 g/dL, daprodustat therapy is ceased until the haemoglobin concentration <11.5 g/dL and therapy is commenced at one dose step lower;
b) where the haemoglobin concentration is in the range ≥9.5 to <11.5 g/dL, the dose is maintained;
c) where the haemoglobin concentration is in the range >11 to ≤11.5 g/dL at two consecutive clinic visits and there has been an increase or no change in the haemoglobin concentration since the last visit, the dose is reduced by one dose step;
d) where the haemoglobin concentration is in the range >11.5 to <12 g/dL and there has been a decrease in haemoglobin concentration since the last visit, the dose is maintained;
e) where the haemoglobin concentration is in the range >11.5 to <12 g/dL and there has been an increase or no change in the haemoglobin concentration since the last visit, the dose is reduced by one dose step;
f) where the haemoglobin concentration is in the range ≥9.5 to <10 at two consecutive clinic visits and there has been a decrease or no change in the haemoglobin concentration since the last visit, the dose is increased by one dose step;
g) where the haemoglobin concentration is in the range 7.5 to <9.5 g/dL and there has been an increase in haemoglobin concentration of ≥0.5 g/dL since the last visit, the dose is maintained;
h) where the haemoglobin concentration is in the range 7.5 to <9.5 g/dL and there has been a decrease, no change or an increase of <0.5 g/dL in haemoglobin concentration since the last visit, the dose is increased by one dose step;
i) where the haemoglobin concentration is <7.5 g/dL, the dose is increased by one dose step;
j) where there has been an increase in haemoglobin concentration of >2 g/dL over 4 weeks, or an increase in haemoglobin concentration of >1 g/dL over 2 weeks, the dose is reduced by one dose step; and
k) where there has been a decrease in haemoglobin concentration of >2 g/dL over 4 weeks, or a decrease in haemoglobin concentration of >1 g/dL over 2 weeks, the dose is increased by one dose step.

In one embodiment, the invention provides the immediate release formulation of the invention for use in the treatment of anemia due to chronic kidney disease, wherein the immediate release tablet of the invention is administered once daily at one of the following doses: 1, 2, 4, 6, 8, 12, 16 and 24 mg (dose of free acid) in accordance with a dosage regimen as described herein.

In one embodiment, the invention provides use of a composition of daprodustat or a pharmaceutically acceptable salt thereof which composition comprises less than 0.15% w/w of a compound of formula (II) or a pharmaceutically acceptable salt thereof in the manufacture of the immediate release formulation of the invention for use in the treatment of anemia due to chronic kidney disease, wherein the immediate release formulation of the invention is administered once daily at one of the following doses: 1, 2, 4, 6, 8, 12, 16 and 24 mg (dose of free acid) in accordance with a dosage regimen as described herein.

In one aspect, the invention provides a dosage regimen for the treatment of anemia due to chronic kidney disease for patients on dialysis wherein the immediate release formulation of the invention is administered three times per week with each dose being either 2 mg, 4 mg, 8 mg, 12 mg, 16 mg, 24 mg, 32 mg or 48 mg and wherein the dose is increased or decreased by one dose step based on the haemoglobin concentration of the patient to maintain the haemoglobin concentration of the patient within the range 10-12 g/dL. In one embodiment, the dose is increased or decreased by one dose step based on the haemoglobin concentration of the patient to maintain the haemoglobin concentration of the patient within the range 10-11 g/dL.

In particular embodiments, the haemoglobin concentration of the patient is monitored at least once every three months. In more particular embodiments, the haemoglobin concentration of the patient is monitored monthly or every four weeks. The skilled person will appreciate that monitoring may be more frequent when treatment is initiated, with the frequency of monitoring decreasing once the haemoglobin concentration of the patient has stabilised within the target range (10 to 12 g/dL or 10 to 11 g/dL).

In embodiments when there is a rapid increase in the haemoglobin concentration of the patient (e.g., exceeding 2.0 g/dL within 4 weeks), the dose is reduced by one dose step or interrupted.

In embodiments where the haemoglobin concentration of the patient exceeds the top end of the target range, the dose is interrupted until the haemoglobin concentration is in target range, and treatment is re-started at one dose level lower.

Clinical judgement is also important in dose increases and reductions. In embodiments where the patient is above the target range and at risk of thromboembolism (e.g. where a patient has had a stroke), the dose is reduced by one dose step or interrupted. In embodiments where the patient is exhibiting symptoms of anemia, the dose is increased by one dose step.

In embodiments where the patient is on dialysis and the patient has previously been treated with an erythropoiesis stimulating agent (ESA), starting doses are based on prior ESA dosage. Table 2 sets out suitable starting doses.

TABLE 2

| Current ESA Dosage | | | | |
|---|---|---|---|---|
| | | methoxy | | |
| Epoetin alfa | Darbepoetin alfa | PEG-epoetin | Daprodustat Dose | |
| IV | SC/IV | beta SC/IV | | Three-times |
| (units/week) | (mcg/4 week) | (mcg /month) | Once daily | per week² |
| <1500 (This dose | | | 2 mg | |
| level is not agreed | | | | |
| yet) | | | | |
| 1500 to 2000 | 20 to 30 | 30 to 40 | 4 mg | 8 mg |
| >2000 to <10000 | >30 to 150 | >40 to 180 | 6 mg | 12 mg |
| ≥10,000 to <20,000 | >150 to 300 | >180 to 360 | 8 mg | 16 mg |
| ≥20,000 | >300 | >360 | 12 mg | 24 mg |

¹ESA: Erythropoiesis-stimulating agent
²Haemodialysis patients only

Table 3 sets out suitable starting doses for patients initiating dialysis not currently treated with an erythropoiesis stimulating agent (ESA).

TABLE 3

| Haemoglobin level (g/dL) | Daprodustat once daily |
|---|---|
| <9 | 4 mg |
| ≥9 to ≤10 | 2 mg |
| >10 | 1 mg |

¹Patients initiating dialysis includes patients in whom dialysis is planned to start in the next 6 weeks, or those who have started dialysis in the last 3 months.
²ESA: Erythropoiesis-stimulating agent

A dosage regimen for treatment of anemia due to chronic kidney disease to maintain haemoglobin concentration in the range 10-11 g/dL is provided, wherein the immediate release tablet of the invention is administered three times per week with each dose being either 2 mg, 4 mg, 8 mg, 12 mg, 16 mg, 24 mg, 32 mg or 48 mg (dose of free acid), and wherein:
a) where the haemoglobin concentration ≥12 g/dL, daprodustat therapy is ceased until the haemoglobin concentration <11.5 g/dL and therapy is commenced at one dose step lower;
b) where the haemoglobin concentration is in the range ≥9.5 to <11.5 g/dL, the dose is maintained;
c) where the haemoglobin concentration is in the range >11 to ≤11.5 g/dL at two consecutive clinic visits and there has been an increase or no change in the haemoglobin concentration since the last visit, the dose is reduced by one dose step;
d) where the haemoglobin concentration is in the range >11.5 to <12 g/dL and there has been a decrease in haemoglobin concentration since the last visit, the dose is maintained;
e) where the haemoglobin concentration is in the range >11.5 to <12 g/dL and there has been an increase or no change in the haemoglobin concentration since the last visit, the dose is reduced by one dose step;
f) where the haemoglobin concentration is in the range ≥9.5 to <10 at two consecutive clinic visits and there has been a decrease or no change in the haemoglobin concentration since the last visit, the dose is increased by one dose step;
g) where the haemoglobin concentration is in the range 7.5 to <9.5 g/dL and there has been an increase in haemoglobin concentration of ≥0.5 g/dL since the last visit, the dose is maintained;
h) where the haemoglobin concentration is in the range 7.5 to <9.5 g/dL and there has been a decrease, no change or an increase of <0.5 g/dL in haemoglobin concentration since the last visit, the dose is increased by one dose step;
i) where the haemoglobin concentration is <7.5 g/dL, the dose is increased by one dose step;
j) where there has been an increase in haemoglobin concentration of >2 g/dL over 4 weeks, or an increase in haemoglobin concentration of >1 g/dL over 2 weeks, the dose is reduced by one dose step; and
k) where there has been a decrease in haemoglobin concentration of >2 g/dL over 4 weeks, or a decrease in haemoglobin concentration of >1 g/dL over 2 weeks, the dose is increased by one dose step.

In one embodiment, the invention provides the immediate release formulation of the invention for use in the treatment of anemia due to chronic kidney disease in patients on dialysis, wherein the immediate release formulation of the invention is administered three times per week with each dose being either: 2 mg, 4 mg, 8 mg, 12 mg, 16 mg, 24 mg, 32 mg or 48 mg (dose of free acid) in accordance with a dosage regimen as described herein.

In one embodiment, the invention provides a composition of daprodustat or a pharmaceutically acceptable salt thereof which composition comprises less than 0.15% w/w of a compound of formula (II) or a pharmaceutically acceptable salt thereof in the manufacture of the immediate release formulation of the invention for use in the treatment of anemia due to chronic kidney disease in patients on dialysis, wherein the immediate release formulation of the invention is administered three times per week with each dose being either: 2 mg, 4 mg, 8 mg, 12 mg, 16 mg, 24 mg, 32 mg or 48 mg (dose of free acid) in accordance with a dosage regimen as described herein.

For the avoidance of doubt, it is noted that any particular dose can be administered in a single dosage form or multiple dosage forms. For example, the dose of 8 mg could be administered as a single 8 mg dosage form, or two 4 mg dosage forms, or four 2 mg dosage forms or eight 1 mg dosage forms, or a 6 mg and a 2 mg dosage form.

It will be apparent that dose adjustments will result in the daprodustat dose being increased or decreased by one dose step at a time. Those receiving the highest (maximum) dose of daprodustat who require a dose increase will maintain the same dose, while those receiving the lowest dose of daprodustat that require a dose decrease will finish daprodustat therapy.

### EXAMPLES

### Example 1

Daprodustat is prepared according to the general synthetic scheme supra in a route comprising Stages 1, 2a, 2b, 2c and 3 wherein P¹ is ethyl and the metal ion is a potassium ion. The impact of impurities derived from Stage 1 of the process on drug substance quality was assessed by determining their purging when intentionally added to the Stage 2/3 process. In a first experiment, impurities generated in Stage 1 (N-cyclohexyl-N-(cyclohexylcarbamoyl)acetamide, 3-(1,3-dicyclohexylureido)-3-oxopropanoic acid, 5-acetyl-1,3-dicyclohexylpyrimidine-2,4,6(1H,3H,5H)-trione and 1,3-dicyclohexyl-2H-pyrano[2,3-d]pyrimidine-2,4,5,7(1H,3H,6H)-tetraone) and Stage 1 starting materials (1,3-dicyclohexylurea and malonic acid) were each spiked at a level of 0.3% w/w in a group into the Stage 1 product 1,3-dicyclohexylpyrimidine-2,4,6(1H,3H,5H)-trione and this was used to produce daprodustat. The levels of each of the spiked materials were identified in daprodustat by HPLC analysis. The only spiked material detectable in daprodustat was 5-acetyl-1,3-dicyclohexylpyrimidine-2,4,6(1H,3H,5H)-trione which was present in daprodustat at a level of 0.8 w/w.

In a second experiment, 0.5% acetic acid was spiked into the Stage 1 product 1,3-dicyclohexylpyrimidine-2,4,6(1H,3H,5H)-trione and this was used to produce daprodustat. This resulted in the presence of 5-acetyl-1,3-dicyclohexylpyrimidine-2,4,6(1H,3H,5H)-trione at a level of 0.09 w/w in daprodustat.

Further studies were performed to determine the impact on drug substance quality, if residual 5-acetyl-1,3-dicyclohexylpyrimidine-2,4,6(1H,3H,5H)-trione and acetic acid were simultaneously present in 1,3-dicyclohexylpyrimidine-2,4,6(1H,3H,5H)-trione (Table 4). Firstly, the impurities were spiked into 1,3-dicyclohexylpyrimidine-2,4,6(1H,3H,5H)-trione independently to quantify their individual impact. 5-acetyl-1,3-dicyclohexylpyrimidine-2,4,6(1H,3H,5H)-trione, when added alone, was confirmed to carry over to daprodustat. Addition of acetic acid at a reduced level caused low but detectable formation of 5-acetyl-1,3-dicyclohexylpyrimidine-2,4,6(1H,3H,5H)-trione. Simultaneous spiking of 5-acetyl-1,3-dicyclohexylpyrimidine-2,4,6(1H,3H,5H)-trione and acetic acid confirmed their synergistic impact on the drug substance.

TABLE 4

| | | 5-acetyl-1,3-dicyclohexylpyrimidine- |
|---|---|---|
| | Level | 2,4,6(1H,3H,5H)-trione present in daprodustat |
| Additive | (w/w) | (% w/w) |
| 5-acetyl-1,3- | 0.3% | 0.21% |
| dicyclohexylpyrimidine- | | |
| 2,4,6(1H,3H,5H)-trione | | |
| Acetic acid | 0.15% | <0.05% |
| 5-acetyl-1,3- | 0.09% | 0.12% |
| dicyclohexylpyrimidine- | 0.15% | |
| 2,4,6(1H,3H,5H)-trione | | |
| Acetic acid | | |
| 5-acetyl-1,3- | 0.07% | 0.11% |
| dicyclohexylpyrimidine- | | |
| 2,4,6(1H,3H,5H)-trione | | |
| Acetic acid | 0.1% | |

This series of experiments demonstrates that levels of 5-acetyl-1,3-dicyclohexylpyrimidine-2,4,6(1H,3H,5H)-trione and acetic acid going into Stage 2 needs to be controlled in order to keep levels of 5-acetyl-1,3-dicyclohexylpyrimidine-2,4,6(1H,3H,5H)-trione below 0.15% in drug product. Levels of 0.1% acetic acid can be tolerated in Stage 2 where levels of 5-acetyl-1,3-dicyclohexylpyrimidine-2,4,6(1H,3H,5H)-trione are also controlled.

### Example 2

Stage 1 comprises reaction of 1,3-dicyclohexylurea and malonic acid in the presence of the dehydrating agent acetic anhydride in acetic acid. The reaction proceeds via an intermediate, 3-(1,3-dicyclohexylureido)-3-oxopropanoic acid. The impact of imidazole on the rate of formation of the desired product and impurities was studied by spiking imidazole at 0.5% w/w in 1,3-dicyclohexylurea. The reaction profile was determined at 6 and 8 h by HPLC analysis of the filtered reaction mixture.

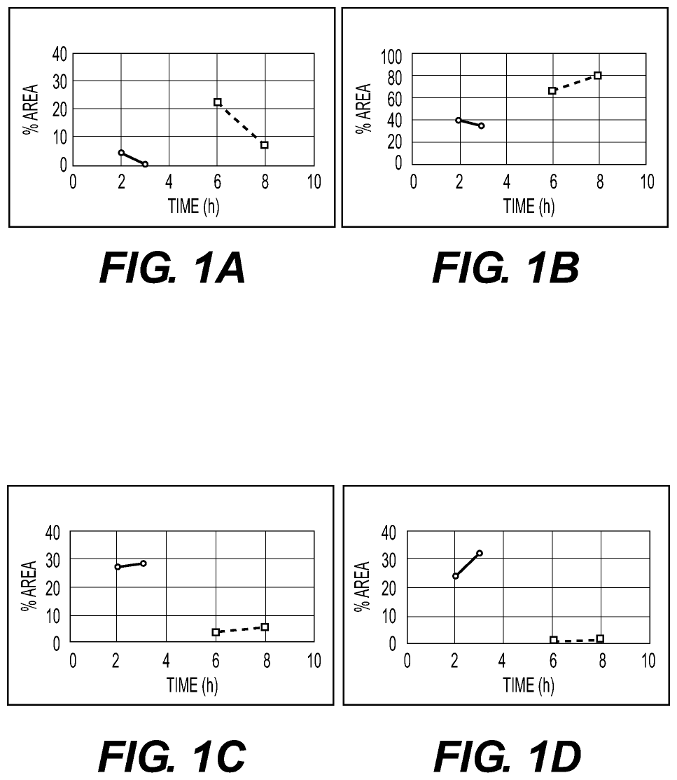

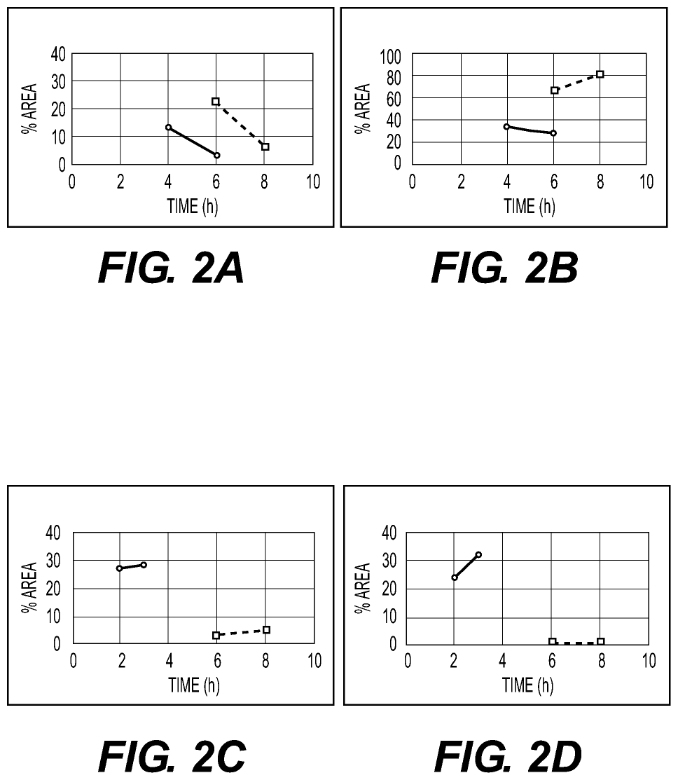

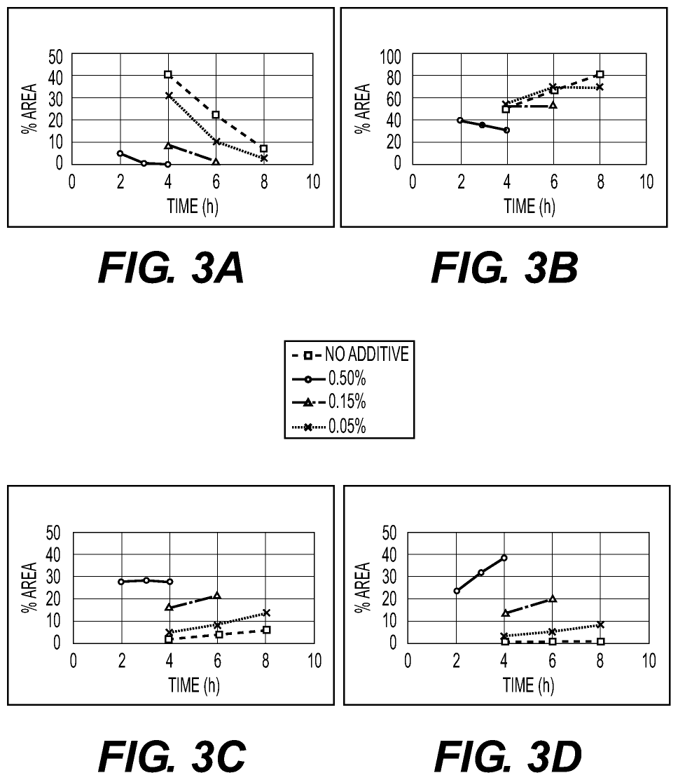

Imidazole significantly increased the rate of intermediate consumption (FIG. 1A) and formation of the product-derived impurities (5-acetyl-1,3-dicyclohexylpyrimidine-2,4,6(1H,3H,5H)-trione (FIG. 1D) and 1,3-dicyclohexyl-2H-pyrano[2,3-d]pyrimidine-2,4,5,7(1H,3H,6H)-tetraone (FIG. 1C)) whilst reducing formation of the 1,3-dicyclohexylpyrimidine-2,4,6(1H,3H,5H)-trione product (FIG. 1B), leading to conversion of the majority of 1,3-dicyclohexylpyrimidine-2,4,6(1H,3H,5H)-trione to a mixture of 5-acetyl-1,3-dicyclohexylpyrimidine-2,4,6(1H,3H,5H)-trione and 1,3-dicyclohexyl-2H-pyrano[2,3-d]pyrimidine-2,4,5,7(1H,3H,6H)-tetraone. The mechanism by which added imidazole causes acceleration of these reactions was inferred from the similarity of the reaction profile when adding exogenous N,N′-dimethylaminopyridine (FIG. 2); both acting as nucleophilic catalysts for the series of condensation reactions occurring in this stage. A study of the reaction progress with varying levels of imidazole added to 1,3-dicyclohexylurea (FIG. 3) showed acceleration of the reaction at loadings as low as 0.05%.

### Example 3

1,3-Dicyclohexylurea is converted to 1,3-dicyclohexylpyrimidine-2,4,6(1H,3H,5H)-trione by treatment with acetic anhydride in acetic acid. Preliminary studies including the study reported in Example 1 showed that 1,3-dicyclohexylpyrimidine-2,4,6(1H,3H,5H)-trione slowly reacts with acetic anhydride as it forms in Stage 1 to give impurity, 5-acetyl-1,3-dicyclohexylpyrimidine-2,4,6(1H,3H,5H)-trione. A screening Design of Experiment (DoE) was conducted to identify parameters and attributes linked to the formation of 5-acetyl-1,3-dicyclohexylpyrimidine-2,4,6(1H,3H,5H)-trione. The study was a two level, six factor, 20 run fractional factorial design with 4 centre points. The parameters and ranges studied are shown in Table 5:

TABLE 5

| Parameter | Low Level | Centre Point | High Level | Units |
|---|---|---|---|---|
| Quantity of acetic | 2.0 | 3.0 | 4.0 | Volumes |
| acid | | | | |
| Quantity of | 1.1 | 1.2 | 1.3 | Molar equivalents |
| malonic acid | | | | |
| Quantity of acetic | 5.0 | 6.0 | 7.0 | Molar equivalents |
| anhydride | | | | |
| Reaction | 50 | 55 | 60 | ° C. |
| temperature | | | | |
| Imidazole content | 0.013 | 0.26 | 0.5 | % w/W |
| in 1,3- | | | | |
| dicyclohexylurea | | | | |
| Power input (at | 24 (150 rpm) | 228 (325 rpm) | 802 (500 rpm) | W/m³ |
| impeller speed) | | | | |

The rate of formation of 5-acetyl-1,3-dicyclohexylpyrimidine-2,4,6(1H,3H,5H)-trione was the response studied. Time course data were collected to determine the initial rate of 5-acetyl-1,3-dicyclohexylpyrimidine-2,4,6(1H,3H,5H)-trione formation under each condition. Samples of the reaction filtrate were taken at regular intervals, and the relative content of 1,3-dicyclohexylpyrimidine-2,4,6(1H,3H,5H)-trione and 5-acetyl-1,3-dicyclohexylpyrimidine-2,4,6(1H,3H,5H)-trione in the filtrate was determined by HPLC. The data for each condition were fitted to a second order polynomial equation to describe the reaction progress. The equation was solved to determine the time when 15% conversion was reached. The initial rate of 5-acetyl-1,3-dicyclohexylpyrimidine-2,4,6(1H,3H,5H)-trione is inverse of this time.

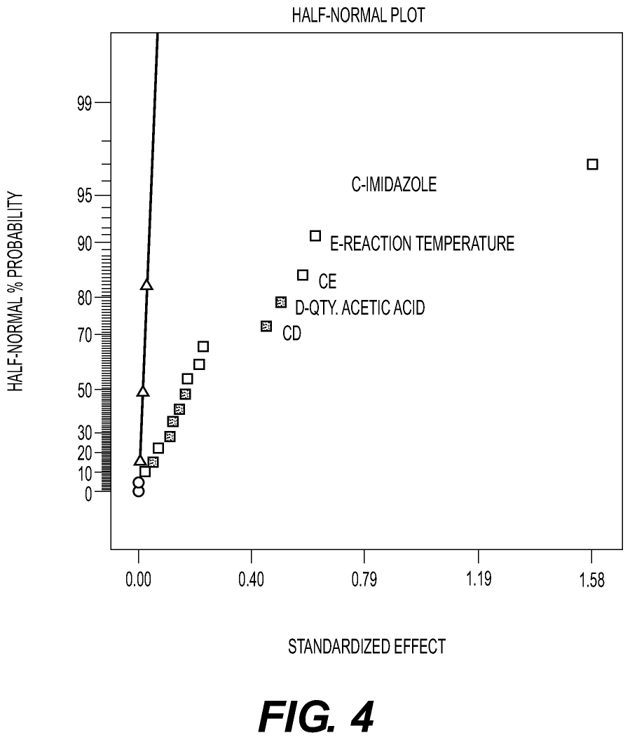

Analysis of the screening study is presented using the half-normal plot in FIG. 4. The imidazole content in 1,3-dicyclohexylurea was found to have a significant (P<0.0001) impact on the rate of 5-acetyl-1,3-dicyclohexylpyrimidine-2,4,6(1H,3H,5H)-trione formation. Reaction temperature (P=0.0003) and the quantity of acetic acid (P=0.0016) also showed effects.

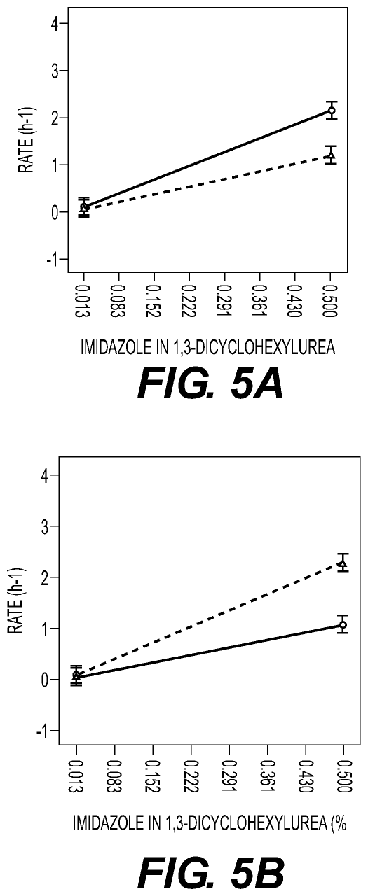

The impact of changes to the quantity of imidazole in 1,3-dicyclohexylurea in combination with quantity of acetic acid or reaction temperature is shown in the interaction plots (FIG. 5). These plots indicate that high quantities of imidazole in 1,3-dicyclohexylurea, in combination with either high reaction temperature or low quantity of acetic acid, resulting in increased reaction concentration, accelerate the formation of 5-acetyl-1,3-dicyclohexylpyrimidine-2,4,6(1H,3H,5H)-trione.

### Example 4

Examples 2 and 3 show that the formation of 5-acetyl-1,3-dicyclohexylpyrimidine-2,4,6(1H,3H,5H)-trione in Stage 1 is dependent on the presence of imidazole in 1,3-dicyclohexylurea and also the quantity of acetic acid and reaction temperature. Example 1 additionally shows that 5-acetyl-1,3-dicyclohexylpyrimidine-2,4,6(1H,3H,5H)-trione cannot be readily purged in later stages. An assessment of the purging capability of the step of isolating the Stage 1 product was performed to establish tolerable limits for this impurity at the end of Stage 1.

The solubility of 5-acetyl-1,3-dicyclohexylpyrimidine-2,4,6(1H,3H,5H)-trione was measured in 29.5% v/v acetic anhydride-acetic acid (a mimic for the solvent composition at the end of Stage 1), acetic acid and water. The solubility was measured by saturation of the solvent with the component, filtration and determination of the component concentration in the supernatant. The solubility of 5-acetyl-1,3-dicyclohexylpyrimidine-2,4,6(1H,3H,5H)-trione in 29.5% v/v acetic anhydride-acetic acid at 15° C. was determined to be 43.7 mg/ml. The solubility of 5-acetyl-1,3-dicyclohexylpyrimidine-2,4,6(1H,3H,5H)-trione in acetic acid at 20° C. was determined to be 62.3 mg/ml and the solubility of 5-acetyl-1,3-dicyclohexylpyrimidine-2,4,6(1H,3H,5H)-trione in water at 25° C. was determined to be <0.1 mg/ml.

A spiking study was performed to determine the purging of 5-acetyl-1,3-dicyclohexylpyrimidine-2,4,6(1H,3H,5H)-trione during isolation of the Stage 1 product, 1,3-dicyclohexylpyrimidine-2,4,6(1H,3H,5H)-trione. 5-acetyl-1,3-dicyclohexylpyrimidine-2,4,6(1H,3H,5H)-trione was added to a suspension of 1,3-dicyclohexylpyrimidine-2,4,6(1H,3H,5H)-trione in 29.5-acetic anhydride-acetic acid. The level of 5-acetyl-1,3-dicyclohexylpyrimidine-2,4,6(1H,3H,5H)-trione following filtration at 15° C. and washing with acetic acid (2×1.0 volumes), followed by water (2×1.0 volumes) is shown in Table 6.

TABLE 6
filtrate

| | | | Level in filtrate | |
|---|---|---|---|---|
| | Amount added | | relative to 1,3- | Isolated 1,3- |
| | relative to 1,3- | Amount | dicyclohexylpyrimidine- | dicyclohexylpyrimidine- |
| | dicyclohexylpyrimidine- | relative | 2,4,6(1H,3H,5H)- | 2,4,6(1H,3H,5H)- |
| | 2,4,6(1H,3H,5H)- | to solvent | trione (% area at | trione purity (% |
| Additive | trione. (% w/w) | (mg/mL) | 210 nm) | w/w) |
| 5-acetyl-1,3- | 19.8 | 45.2 | 75.8 | 100.0 |
| dicyclohexylpyrimidine- | | | | |
| 2,4,6(1H,3H,5H)-trione | | | | |

Process stretching studies were performed to demonstrate the purging of 5-acetyl-1,3-dicyclohexylpyrimidine-2,4,6(1H,3H,5H)-trione at larger scale. The Stage 1 reaction was conducted with the conditions set out in Table 7, that are predicted to generate elevated 5-acetyl-1,3-dicyclohexylpyrimidine-2,4,6(1H,3H,5H)-trione followed by filtration at 15° C. and washing with acetic acid (2×1.0 volumes), followed by water (2×1.0 volumes).

TABLE 7
Reaction conditions

| | |
|---|---|
| Quantity of 1,3-dicyclohexylurea (Molar equivalents) | 1.0 |
| Quantity of acetic acid (Volumes) | 2.7 |
| Quantity of malonic acid (Molar equivalents) | 1.3 |
| Quantity of acetic anhydride (Molar equivalents) | 6.6 |
| Quantity of imidazole in 1,3-dicyclohexylurea (% w/w) | 0.15 |
| Reaction temperature (° C.) | 60 |

HPLC of the filtrate showed that the % area relative to the reaction product, 1,3-dicyclohexylpyrimidine-2,4,6(1H,3H,5H)-trione, was 45.6. The purity of 1,3-dicyclohexylpyrimidine-2,4,6(1H,3H,5H)-trione (% area) was 100% with less than <0.5% (% area) total impurities.

### Example 5

Tablet formulations of daprodustat free acid may be prepared as follows. The tablet cores comprise granules and extragranular components. Granules are prepared by adding daprodustat, mannitol, microcrystalline cellulose, hypromellose 2910 and croscarmellose sodium into a high shear granulator. The powders are blended under high shear for at least 5 minutes and granulation performed while spraying at least 26% w/w purified water over a water addition time of at least 7 minutes and wet massing time of at least 2 minutes. The wet granules are dried in a fluid bed dryer to a target moisture content of not exceeding 2% w/w at a product temperature of at least 38° C. and the granules are dry milled to normalize granule size distribution. The milled granules are futher blended with extragranular components mannitol, microcrystalline cellulose, croscarmellose sodium and glidant colloidal silicon dioxide. Magnesium stearate is added and the resulting mixture is compressed using compaction pressures in the range 180 to 370 MPa into tablet cores using a rotary tablet press under the following conditions.
Tablet shape/size: round, biconvex tablets/7 mm diameter (≤4 mg); 9 mm diameter (≥6 mg)
Compression speed of at least 40000 tablets per hour

The compositions of the tablets are provided in Table 8.

TABLE 8

| | Quantity (mg/tablet) | | | | |
|---|---|---|---|---|---|
| Component | 1 mg | 2 mg | 4 mg | 6 mg | 8 mg |

Granules

| | | | | | |
|---|---|---|---|---|---|
| Daprodustat | 1.00 | 2.00 | 4.00 | 6.00 | 8.00 |
| Mannitol | 72.30 | 71.60 | 70.22 | 141.83 | 140.45 |
| Microcrystalline Cellulose | 31.88 | 31.58 | 30.96 | 62.54 | 61.92 |
| Hypromellose 2910 | 5.63 | 5.63 | 5.63 | 11.25 | 11.25 |
| Croscarmellose Sodium | 1.69 | 1.69 | 1.69 | 3.38 | 3.38 |
| Purified Water | — | — | — | — | — |

Extragranular Components

| Mannitol | 20.44 | 20.44 | 20.44 | 40.87 | 40.87 |
|---|---|---|---|---|---|
| Microcrystalline Cellulose | 10.50 | 10.50 | 10.50 | 21.00 | 21.00 |
| Croscarmellose Sodium | 4.50 | 4.50 | 4.50 | 9.00 | 9.00 |
| Colloidal Silicon Dioxide | 0.56 | 0.56 | 0.56 | 1.13 | 1.13 |
| Magnesium Stearate | 1.50 | 1.50 | 1.50 | 3.00 | 3.00 |
| Core tablet weight | 150.0 | | | 300.0 | |
| (mg) | | | | | |

Purified water for granulation is removed during processing and does not remain in the tablet.

1-27. (canceled)

28. A process for preparing 1,3-dicyclohexylpyrimidine-2,4,6(1H,3H,5H)-trione comprising reacting 1,3-dicyclohexylurea with malonic acid, wherein the products of the reaction are 1,3-dicyclohexylpyrimidine-2,4,6(1H,3H,5H)-trione and a compound of formula (II) 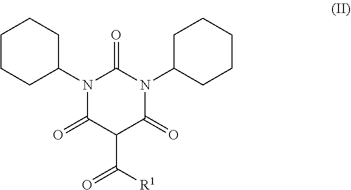
wherein R¹ is C₁₋₆alkyl, wherein the solvent system is selected to maintain the compound of formula (II) in solution at the temperature at which 1,3-dicyclohexylpyrimidine-2,4,6(1H,3H,5H)-trione is isolated, and wherein the reaction is terminated before the concentration of formula (II) exceeds its solubility in the solvent system employed.

29. A process for preparing 1,3-dicyclohexylpyrimidine-2,4,6(1H,3H,5H)-trione according to claim 28, wherein the compound of formula (II) is 5-acetyl-1,3-dicyclohexylpyrimidine-2,4,6(1H,3H,5H)-trione.

30. A process for preparing 1,3-dicyclohexylpyrimidine-2,4,6(1H,3H,5H)-trione according to claim 29, wherein the solvent system is 29.5% v/v acetic anhydride-acetic acid, the isolation temperature is 15° C., and the reaction is terminated before the concentration of 5-acetyl-1,3-dicyclohexylpyrimidine-2,4,6(1H,3H,5H)-trione reaches 45.2 mg/ml.

31. A process for preparing 1,3-dicyclohexylpyrimidine-2,4,6(1H,3H,5H)-trione according to claim 2, wherein when the reaction uses 1,3-dicyclohexylurea (1.0 wt), malonic acid (1.3 wt), acetic acid (2.7 wt) and acetic anhydride (6.6 eq) and the isolation temperature is 15° C., the reaction should be terminated before the percentage of 5-acetyl-1,3-dicyclohexylpyrimidine-2,4,6(1H,3H,5H)-trione exceeds 45% by area of the products of the reaction.

32. A process for preparing 1,3-dicyclohexylpyrimidine-2,4,6(1H,3H,5H)-trione according to claim 28, wherein the reaction is terminated by filtration.

33. A process for preparing 1,3-dicyclohexylpyrimidine-2,4,6(1H,3H,5H)-trione according to claim 32, wherein the filter cake is washed with not less than 2 volumes of a solvent other than a carboxylic acid of formula R¹CO₂H.

34. A process for preparing 1,3-dicyclohexylpyrimidine-2,4,6(1H,3H,5H)-trione according to claim 28, wherein the 1,3-dicyclohexylurea starting material contains not greater than 0.05% (w/w) imidazole.

35. A process for preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a step of preparing 1,3-dicyclohexylpyrimidine-2,4,6(1H,3H,5H)-trione as defined in claim 28.

36. A process for preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof according to claim 35, wherein the levels of a carboxylic acid of formula R¹CO₂H in 1,3-dicyclohexylpyrimidine-2,4,6(1H,3H,5H)-trione are less than or equal to 0.1% (w/w) immediately prior to reaction with a compound of formula (III).

37. A process for preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof according to claim 36, wherein said N-[(1,3-dicyclohexyl-6-hydroxy-2,4-dioxo-1,2,3,4-tetrahydro-5-pyrimidinyl)carbonyl]glycine or a pharmaceutically acceptable salt thereof is in crystalline form.

38. A composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof, wherein N-[(1,3-dicyclohexyl-6-hydroxy-2,4-dioxo-1,2,3,4-tetrahydro-5-pyrimidinyl)carbonyl]glycine or a pharmaceutically acceptable salt thereof is obtained according to a process as defined in claim 35.

39. A process for preparing an immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a step of preparing N-[(1,3-dicyclohexyl-6-hydroxy-2,4-dioxo-1,2,3,4-tetrahydro-5-pyrimidinyl)carbonyl]glycine or a pharmaceutically acceptable salt thereof as defined in claim 35.

40. An immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof obtained by the process of claim 39.

41. An immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof, comprising a composition of N-[(1,3-dicyclohexyl-6-hydroxy-2,4-dioxo-1,2,3,4-tetrahydro-5-pyrimidinyl)carbonyl]glycine or a pharmaceutically acceptable salt thereof which composition comprises less than 0.15% w/w of a compound of formula (II) or a pharmaceutically acceptable salt thereof relative to daprodustat drug substance.

42. An immediate release formulation of N-[(1,3-dicyclohexyl-6-hydroxy-2,4-dioxo-1,2,3,4-tetrahydro-5-pyrimidinyl)carbonyl]glycine or a pharmaceutically acceptable salt thereof according to claim 41, wherein the compound of formula (II) or a pharmaceutically acceptable salt thereof is 5-acetyl-1,3-dicyclohexylpyrimidine-2,4,6(1H,3H,5H)-trione.

43. A method for the treatment of anemia due to chronic kidney disease in a subject in need thereof, comprising administering to said subject the immediate release formulation as defined claim 41.

44. A method according to claim 43, wherein the subject is iron deficient and wherein the subject additionally receives supplemental iron therapy.

45. A method according to claim 43, wherein the immediate release formulation is administered once daily at a dose of either 1 mg, 2 mg, 4 mg, 6 mg, 8 mg, 12 mg, 16 mg or 24 mg and wherein the dose is increased or decreased by one dose step based on the haemoglobin concentration of the patient to maintain the haemoglobin concentration of the patient within the range 10-11 g/dL.

46. A method according to claim 45, wherein the patient is not on dialysis.

47. A method according to claim 43, wherein the immediate release formulation is administered three times per week with each dose being either 2 mg, 4 mg, 8 mg, 12 mg, 16 mg, 24 mg, 32 mg or 48 mg and wherein the dose is increased or decreased by one dose step based on the haemoglobin concentration of the patient to maintain the haemoglobin concentration of the patient within the range 10-11 g/dL.

48. A method according to claim 45, wherein the patient is on dialysis.

49. An immediate release formulation for use according to claim 48, wherein the haemoglobin concentration of the patient is monitored at least once every three months.

50. An immediate release formulation for use according to claim 48, wherein when there is an increase in haemoglobin concentration of the patient exceeding 2.0 g/dL within 4 weeks, the dose is reduced by one dose step or interrupted.

51. A compound of formula (II) or a pharmaceutically acceptable salt thereof: 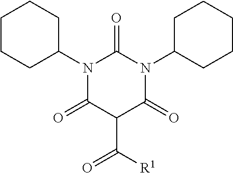
wherein R¹ is C₁₋₆alkyl.

52. A compound that is 5-acetyl-1,3-dicyclohexylpyrimidine-2,4,6(1H,3H,5H)-trione.
